(19) United States
(12) Patent Application Publication
(10) Pub. No.: US 2022/0000539 A1
(43) Pub. Date: Jan. 6, 2022

(54) RF ELECTROSURGICAL TISSUE SEALING SYSTEM AND METHOD

(71) Applicant: Intuitive Surgical Operations, Inc., Sunnyvale, CA (US)

(72) Inventors: Jignesh M. Shah, Sunnyvale, CA (US); Duane W. Marion, Scottsdale, AZ (US)

(21) Appl. No.: 17/291,275

(22) PCT Filed: Oct. 31, 2019
(86) PCT No.: PCT/US2019/059253

§ 371 Date: May 4, 2021

Related U.S. Application Data
(60) Provisional application No. 62/756,995, filed on Nov. 7, 2018.

(51) Int. Cl. A61B 18/14; A61B 18/12

(52) U.S. Cl. CPC A61B 18/1445; A61B 2018/0063; A61B 18/1206

(57) ABSTRACT

Abstract: An electrosurgical method is provided to seal biological tissue comprising providing RF power to the tissue during an RF power delivery time interval comprising: measuring a change in impedance of the tissue with respect to RF energy delivered to the tissue during an initial portion of the time interval; selecting an RF power delivery profile, based at least in part upon the measured change in impedance of the tissue with respect to RF energy delivered to the tissue; and imparting RF power to the tissue according to the selected RF power delivery profile during a latter portion of the time interval.

### CLAIM OF PRIORITY

This application claims the benefit to U.S. Patent Application Ser. No. 62/756,995, filed on Nov. 7, 2018, which is incorporated by reference herein in its entirety.

### BACKGROUND

Electrosurgery involves the use of electricity to generate heat within biological tissue to cause thermal tissue effects resulting in sealing or resulting in incision and removal of the tissue through one or more of desiccation, coagulation, or vaporization, for example. Benefits include the ability to make precise cuts with limited blood loss. Electrosurgical devices are frequently used during surgical procedures to help prevent blood loss in hospital operating rooms or in outpatient procedures. Electrosurgery typically involves using radio frequency (RF) alternating current (AC) that creates heat by resistive heating as the current passes through the tissue.

An electrosurgical sealing process imparts RF energy to biological tissue for a time interval and at an energy level sufficient to cause sealing of the tissue without imparting clinically significant damage to surrounding tissue. An electrosurgical cutting process imparts RF energy to biological tissue for a time interval and at an energy level sufficient to cause cutting of the tissue without imparting clinically significant damage to surrounding tissue. The delivery of RF energy during a sealing or cutting process typically halts when effective sealing or cutting has been achieved.

A typical electrosurgical signal generator uses a multi-stage voltage converter to convert AC line power to a controlled high frequency signal required to perform an electrosurgical procedure. This approach ordinarily includes converting an AC line input to direct current (DC) signal and converting the DC signal to an RF signal. The RF output is imparted to electrodes at a surgical instrument end effector that a surgeon manipulates to impart high frequency energy to seal or cut anatomical tissue. A typical prior electrosurgical instrument includes a pair of opposing first and second jaws that are movable relative to one another from a first spaced apart position to a second position for grasping tissue therebetween. Each jaw includes an electrode configured to contact a tissue surface captured between the jaws. An RF electrosurgical signal source conducts RF current through tissue disposed between the jaws to seal and/or cut vessels within the tissue.

An electrosurgical sealing activity often is used to initially seal a target tissue, immediately followed by a transection activity that transects (cuts) the sealed tissue. Thus, tissue vessels are sealed before they are cut. A previous surgical instrument has been provided, for example, that uses RF sealing electrodes to seal adjacent sections of tissue on opposite side of a tissue region to be cut, in combination with a mechanical blade instrument configured to transect the sealed tissue sections. Another previous surgical instrument has been provided, for example, that uses RF sealing electrodes to seal adjacent sections of tissue on opposite side of a tissue region to be cut, in combination with RF is cutting electrodes to transect the sealed tissue sections.

During electrosurgical sealing activity, an RF electrical signal is conducted through tissue disposed between the electrodes of the first and second jaws. During tissue sealing, RF current density between electrodes is selected to achieve a rate of tissue heating to result in sealing, which as used herein refers to tissue dehydration, vessel wall shrinkage and coagulation of blood constituents and collagen denaturatization and bonding. During tissue cutting, a higher RF current density between electrodes is selected to achieve a rate of tissue heating to result in plasma discharge, which results in cutting tissue, which as used herein refers to dissecting of tissue through vaporization, for example. It is noted that although RF electrosurgical sealing signals and RF electrosurgical cutting signals often deliver the same power, they ordinarily use different voltage and current levels to do so.

RF sealing typically is used on a variety of different biological tissue types, including skeletonized individual vessels, tissue bundles and thin, vascularized tissues such as mesentery. Mesentery typically has a lower initial impedance than an unskeletonized artery, that is, an artery which has not been dissected out from its surrounding tissue bundle. These different types of tissue structures require varying amounts of total energy and a differing activation times to create an effective seal, with tissue bundles and mesentery typically requiring more energy and/or longer activation times. An ineffective seal can result in seepage of blood at the site of the seal, or in the case of bundled or mesentery tissue, cases where not all the vessels within the bundle being completely sealed. Previous RF sealing instruments typically rely on the monitoring of electrical parameters such as voltage, current, power, phase angle or impedance, during the RF sealing activity, to determine when the energy delivery has reached a threshold and should be ended. This approach generally is effective. However, different types of tissues require different amounts of RF energy to achieve an effective seal, and therefore, the energy delivery thresholds suitable for some tissues are not suitable for other tissues. Thus, there is a need for a system and method to that takes into account biological tissue type to determine the amount of RF energy to deliver to seal the tissue.

### SUMMARY

In one example aspect, an electrosurgical method is provided to seal biological tissue. RF power is imparted to the tissue during an RF power delivery time interval. A change in impedance of the tissue with respect to RF energy delivered to the tissue is measured during an initial portion of the RF power delivery time interval. An RF power delivery profile is selected to impart RF power to the tissue during a latter portion of the RF power delivery time interval following the initial portion, based at least in part upon the measured change in impedance of the tissue with respect to RF energy delivered to the tissue during the initial portion. RF power is imparted to the tissue according to the selected RF power delivery profile during the latter portion of the RF power delivery time interval.

In another example aspect, an electrosurgical method is provided to seal biological tissue. RF power is imparted to the tissue during an RF power delivery time interval. RF current within the tissue is measured during an initial portion of the RF power delivery time interval. An RF power delivery profile is selected to impart RF power to the tissue during a latter portion of the RF power delivery time interval following the initial portion, based at least in part upon the measured RF current within the tissue during the initial portion. RF power is imparted to the tissue according to the selected RF power delivery profile during the latter portion of the RF power delivery time interval.

The scope of the invention is defined by the claims, which are incorporated into this section by reference. A more complete understanding of examples of the present invention will be afforded to those skilled in the art, as well as a realization of additional advantages thereof, by a consideration of the following detailed description of one or more examples. Reference will be made to the appended sheets of drawings that will first be described briefly.

### BRIEF DESCRIPTION OF DRAWINGS

FIG. 1 is an illustrative block diagram representing an example electrosurgical generator system.

FIG. 2 is an illustrative side view of a portion of an example electrosurgical instrument end effector that includes a pair of opposed jaws that include a corresponding pair of opposed electrodes.

FIG. 3A is an illustrative drawing representing an example high impedance first tissue portion disposed between the example jaws of FIG. 2.

FIG. 3B is an illustrative drawing representing an example second tissue portion disposed between the example jaws of FIG. 2.

FIG. 3C is an illustrative drawing representing an example third tissue portion disposed between the example jaws of FIG. 2.

FIG. 4A is a flowchart illustrating an example method of RF energy delivery during initial and latter portions of a biological tissue sealing process based upon tissue impedance measurement during the initial portion of the sealing process.

FIG. 4B is a flowchart illustrating a method of RF energy delivery during initial and latter portions of a biological tissue sealing process based upon phase angle between RF voltage and RF current during an initial portion of the sealing process.

FIG. 5 is a flowchart illustrating an example method of RF energy delivery during initial and latter portions of a biological tissue sealing process based upon RF current flow through tissue during an initial portion of the sealing process.

FIGS. 6A-6B are illustrative example power delivery profiles representing alternative example methods to control RF energy imparted through biological tissue in response to determination during an initial window of a lower impedance tissue type.

FIGS. 7A-7C are illustrative alternative example power delivery profiles representing alternative example methods to control RF energy imparted through biological tissue in response to determination during an initial window of a higher impedance tissue type.

FIG. 8 is an illustrative example plot showing tissue impedance versus time during RF power delivery to a lower impedance artery tissue.

FIG. 9 is an illustrative example plot showing tissue impedance versus time during RF power delivery to a higher impedance thin mesentery tissue.

FIGS. 10A-10B are illustrative plots showing impedance versus time (FIG. 10A) and current versus time (FIG. 10B) during RF power delivery to tissues having three different impedance types.

Examples of the present invention and their advantages are best understood by referring to the detailed description that follows. It should he appreciated that like reference numerals are used to identify like elements illustrated in one or more of the figures. It should also be appreciated that the figures may not be necessarily drawn to scale.

### DETAILED DESCRIPTION

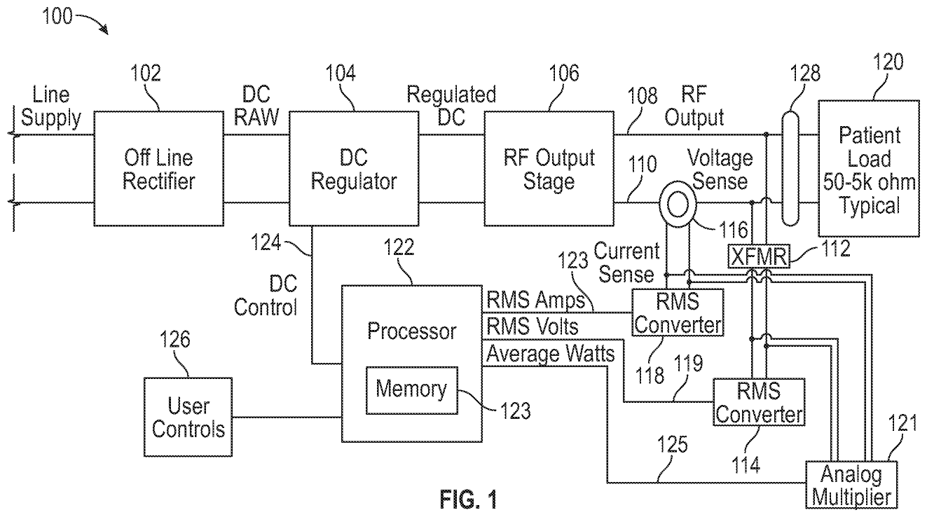

FIG. 1 is an illustrative block diagram representing an example electrosurgical generator system 100. The system 100 includes a processor 122 that includes non-transitory memory to store instructions, an off line rectifier 102 coupled to convert an AC line voltage signal to a raw rectified DC signal, a DC regulator 104 to convert the rectified DC voltage signal to a controlled DC voltage signal, and an RF output stage 106 coupled to convert the controlled DC voltage to high frequency output energy that can be applied across first and second output terminals 108, 110 at a surgical instrument end effector (not shown). Voltage monitoring circuitry includes a voltage transformer 112 and a first RMS converter 114 coupled to monitor an RF output voltage across the first and second output terminals 108, 110 and to provide an RMS voltage value via line 119 to the processor 122. Current monitoring circuitry includes a current sense transformer 116 and a second RMS converter 118 coupled to monitor an RF output current between the first and second output terminals 108, 110 and to provide via line 123 an RMS current to the processor 122. An analog multiplier circuit 121 is coupled to determine real RF output power based upon the product of the instantaneous values of the RF output voltage and analog RF Output current and to provide via line 125 a DC representation of the real power value to the processor 122. A user input control 126 is coupled to receive user input parameters to the processor 122, which may include a maximum high frequency current, voltage or power, a target high frequency voltage, high frequency current or high frequency power, or some combination of these values, for example.

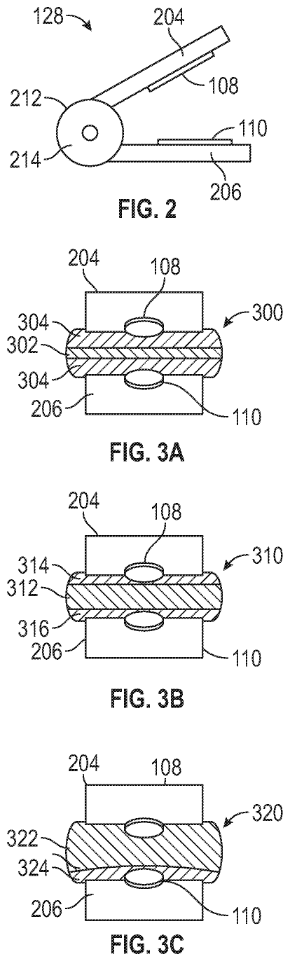

In operation, the first and second output terminals 108, 110 may be disposed at a surgical instrument end effector 128 to contact two different locations on biological tissue 120. The RF output voltage may represent voltage across the biological tissue 120 between the first and second terminals 108, 110 and the RF output current may represent current passing through the biological tissue 120 between the first and second terminals 108, 110. In some examples, the first and second terminals 108, 110 are disposed at first and second articulated jaws, described below with reference to FIGS. 2-3C, configured to grip biological tissue between them. A processor 122 provides a voltage control signal on control line 124 to the DC regulator 104 to determine the controlled. voltage based at least in part upon at least one of the RF output voltage and the RF output current and the RF output power and based upon user input received via a user input control block.

During a surgical procedure the voltage control signal may be varied based at least in part upon variations in impedance and based at least in part upon variations in current measured between the first and second output terminals 108, 110. In general, the impedance load of a patient's biological tissue 120 typically can range from 50 to 5 ohms, depending on the electrosurgical device used and tissues being targeted. The first RMS converter 114 converts a sensed RF output voltage signal to a first DC feedback signal indicating an RF output voltage level. The second RMS converter 118 converts the sensed RF output current signal to a second DC feedback signal indicating an RF output current level. The analog multiplier circuit 121 converts the sensed RF output voltage and the sensed RF output current to third DC feedback signal indicating average real RF output power. The processor 122 produces a voltage control signal on line 124 to cause the DC regulator 104 to produce a controlled DC voltage level based upon at least in part upon one of the sensed RF output voltage, the sensed RF output current and the RF output power. Additional details of an example electrosurgical generator system 100 are provided in Provisional Patent Application Ser. No. 62/513,287, filed May 31, 2017, which is expressly incorporated herein in its entirety by this reference.

A determination of a value for the voltage control signal on line 124 may be based upon the well-known relationship,

P=V²/Z

In some examples, a value for the voltage control signal may be based upon an algorithm such as a Proportional-Integral-Derivative control loop. A target RF power level may be user-specified or dependent upon a surgical procedure, for example.

During a surgical procedure, the tip of an electrosurgical instrument may move between different tissue portions having different impedances. Typically, the surgical effect of the electrosurgical instrument is directly related to the power delivered to the tissue. Accordingly, RF power is regulated in order to maintain a consistent surgical effect as the instrument is moved by a surgeon between different tissue types. In general, the higher the impedance of the tissue the higher the RF voltage required to provide a desired therapeutic effect at the electrosurgical instrument tip. Conversely, the lower the impedance of the tissue the lower the RF voltage required to provide a desired therapeutic effect at the electrosurgical instrument tip. Providing a voltage that is too high to biological tissue may result in excessive power delivery that may result in excessive tissue heating and unwanted tissue damage. Moreover, during a surgical procedure, an electrosurgical instrument tip may move into and out of contact with patient tissue such as when the tip is momentarily suspended in air above patient tissue, for example. The impedance between the first and second terminals while the tip is suspended in air approaches infinity.

FIG. 2 is an illustrative side view of a portion of an example the electrosurgical instrument end effector 128 of FIG. 1 that includes a pair of opposed jaws 204, 206 that include a corresponding pair of opposed electrodes 108, 110. The jaws 204, 206 are mounted to a support structure 212 that includes a pivot axis 214. At least one of the jaws is pivotally mounted to be operable rotate relative to the pivot axis 214 to transition the end effector 128 between an ‘open’ configuration illustrated in FIG. 2, in which the jaws 204, 206 are spaced farther apart from one another, and a ‘closed’ configuration illustrated below with reference to FIGS. 3A-3C, in which the jaws 204, 206 are spaced closer together such that biological tissue 216 may be grasped between them.

The inventors observed that different biological tissue compositions have different characteristic impedance levels and different characteristic rises in impedance during initial application of electrosurgical RF sealing energy. More specifically, the initial impedance and the initial rate of change of impedance are different for different tissue types in response to the same RF energy input during the initial application of RF sealing energy. For example, the initial rise in tissue impedance of tissue that includes multiple vascularized elements such as in mesentery, typically is slower than the initial rise in tissue impedance of tissue that includes only a single vessel. Moreover, the inventors observed, for example, that at the time of initial application of RF energy, mesentery typically has a lower initial impedance than an unskeletonized artery.

The inventors realized that in view of the differences in rates of impedance change for different tissue types and the differences in initial impedance values for different tissue types, monitoring the electrical characteristics of tissue during electrosurgical sealing to determine when the energy delivery has reached a threshold and should be ended, without determining the initial impedance characteristics of the tissue, can be insufficient to ensure effective sealing of some tissue types. Moreover, the inventors realized that merely seeking to ensure effective tissue sealing by imparting extra RF electrosurgical sealing energy, beyond what ordinarily is required to achieve effective sealing, can lead to unwanted collateral damage to surrounding tissue.

The inventors realized that different electrosurgical sealing scenarios generally require different amounts of energy to achieve effective sealing. Examples of electrosurgical sealing scenarios that typically require a greater amount of RF sealing energy include sealing high impedance tissues such as fatty tissues and/or unskeletonized vessels and sealing larger tissue bites grasped between instrument jaws. Examples of electrosurgical sealing scenarios typically require a lesser amount of sealing energy include sealing lower impedance tissues such as mesentery and omentum and well-dissected vessels and sealing smaller tissue bites within the instrument jaws.

FIG. 3A is an illustrative drawing representing an example high impedance first tissue portion 300 disposed between the first and second jaws 204, 206. The example first tissue portion 300 includes thinner lower impedance tissue portion 302 such as mesentery sandwiched between two thicker higher impedance tissue type portions 304, such as fatty tissue. Each of the higher impedance tissue portions is in electrical contact with one of the electrodes 108, 110. FIG. 3B is an illustrative drawing representing an example second tissue portion 310 disposed between the first and second jaws 204, 206. The example second tissue portion 310 includes a thicker lower impedance tissue portion 312 such as mesentery tissue sandwiched between two thinner higher impedance tissue type portions 314, such as fatty tissue. FIG. 3C is an illustrative drawing representing an example third tissue portion 320 disposed between the first and second jaws 204, 206. The example third tissue portion 320 includes a thicker lower impedance tissue portion 322, such as mesentery, and a thinner higher impedance tissue type portion 324, such as fatty tissue. It is noted that the jaws 204, 206 are spaced closer together in grasping the example third tissue portion 320 of FIG. 3A, than they are in grasping the first and second tissue portions 300, 310 of FIGS. 3B-3C, since the example third tissue portion 320 is thinner than the example first and second tissue portions 300, 310. Thus, it will be appreciated that the example, first, second, and third tissue portions 300, 310, 320 present three different types of electrosurgical sealing scenarios in terms of tissue type constituents, e.g., proportion of high impedance and low impedance tissue types, and/or in terms of tissue dimensions, e.g., tissue thickness between the jaws 204, 206.

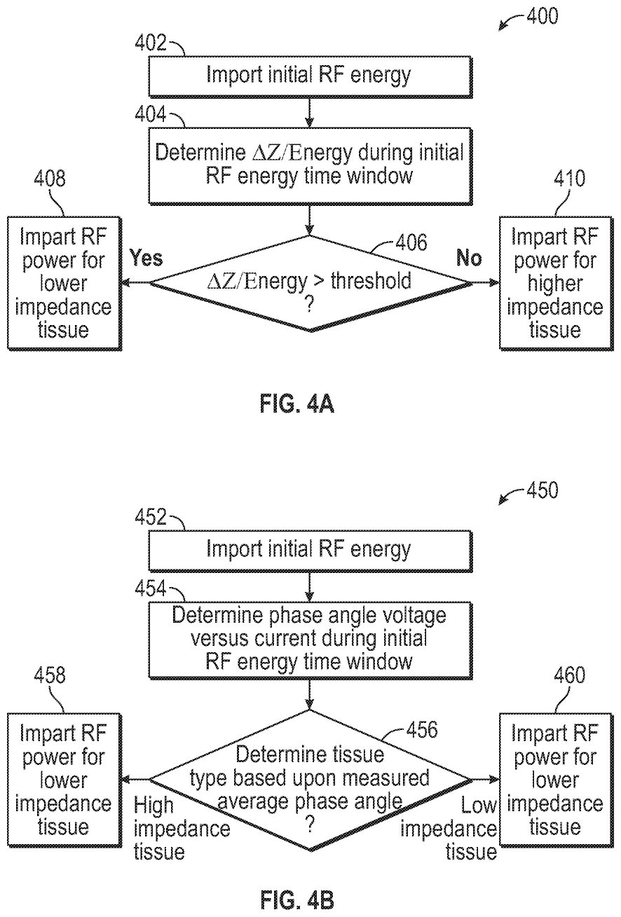

FIG. 4A is a flowchart illustrating n example method 400 of RF energy delivery during initial and latter portions of a biological tissue sealing process based upon tissue impedance measurement during the initial portion of the sealing process. Operations in the method 400 may be performed using components of the electrosurgical generator system 100. As shown in FIG. 4A, the method 400 includes operations 402-410.

In operation 402, the end effector 128 imparts RF power to biological tissue in range 0 to 500 ohms during an initial impedance measurement time window, which has a duration in a range 1 to 1000 ms. The Specifically, while the jaws 204, 206 of the end effector 128 grip biological tissue between them, as shown in the illustrative examples of FIGS. 3A-3C, the RF output stage 106 produces an RF voltage across the electrodes 108, 110, which causes RF current flow between through biological tissue. RF power level during the initial impedance measurement time window is sufficient to start sealing biological tissue grasped between the jaws 204, 206. In the example process 400, RF power during the initial impedance measurement me window, RF power level is in a range 1-50 Watts.

In operation 404, the processor 122 executes instructions stored in memory 123 to cause the processor to determine a change in impedance of the tissue with respect to energy delivered during the initial impedance measurement time window.

ΔZ_{mw}/E_{mw}

where ΔZ_{mw} represents change in impedance during the initial impedance measurement time window, and E_{mw} represents energy delivered during the initial impedance measurement time window. In one example of the method 400, the processor 122 can vary power delivery to the tissue during the initial time window. The operation 404 totalizes the power during the initial time window to determine change in impedance with respect to energy. An example of totalizing is performing an integration. Another example of totalizing is performing a running average. It will be understood that energy can be computed as the product of power multiplied by time. In another example of the of the method 400, the micro controller 122 delivers predetermined power to the tissue during the initial time window eliminating the need to totalize the power delivery.

The processor 122 obtains RF voltage samples from the voltage transformer 112 and first RMS converter 114, which 114 monitor RF output voltage across the first and second electrodes 108, 110 during the initial impedance measurement time window. The processor 122 obtains RF current samples from the current sense transformer 116 and a second RMS converter 118, which monitor an RF output current between the first and second electrodes 108, 110 during the initial impedance measurement time window. The processor 122 obtains RF current samples from the analog multiplier circuit 121, which determines RF power delivered to biological tissue between the electrodes 108, 110 during the initial impedance measurement time window.

In decision operation 406, the processor 122 executes instructions stored in memory 123 to cause the processor to determine tissue type based upon change in impedance with respect to energy delivered during the initial impedance measurement time window. In some examples of method 400, operation 406 determines whether change in impedance with respect to energy meets a threshold. An example method 400 determines tissue type according to rules set forth in the Table A.

TABLE A

| ΔZ_{mw}/E_{mw} for initial impedance | |
|---|---|
| measurement time window of tissue | Parameters for latter portion of |
| sealing process | tissue sealing process |
| If ΔZ_{mw}/E_{mw} in a range 10%/25 J | Use lower impedance tissue |
| | parameters |
| IF ΔZ_{mw}/E_{mw} in a range 25%/25 J | Use higher impedance tissue |
| | parameters |

It will be appreciated that decision operation 406 determines tissue type in terms of impedance characteristics of the tissue rather than precise identification of the biological constituents of the tissue. Different tissue samples may have different combinations of high and low impedance constituents. Rather than identify the specific tissue constituents, the operation 406 determines a measure indicative of impedance of a tissue portion captured between the jaws 204, 206.

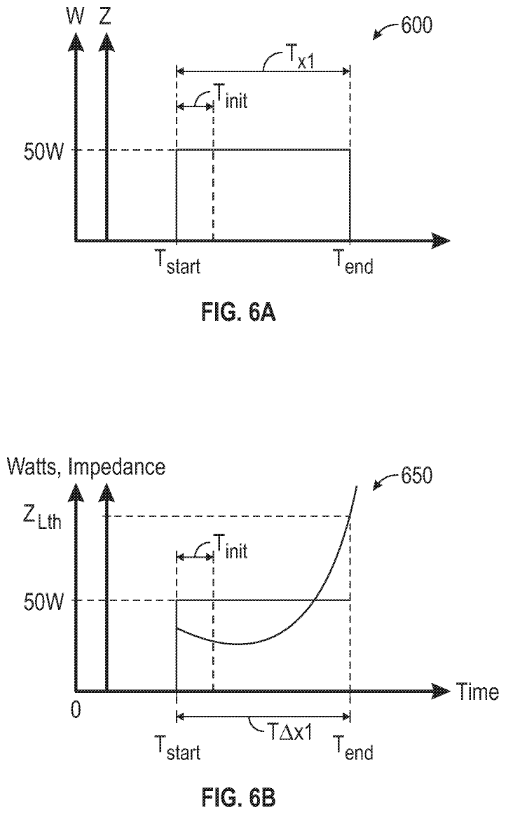

During operation 408, in response to decision operation 406 determining to use lower impedance tissue parameters during a latter portion of the tissue sealing process, the processor 122 executes instructions stored in memory 123 to cause the processor to provide control signals to the DC regulator 104 to cause the RF output stage 106 to imparts RF energy to tissue electrically coupled between the electrodes 108, 110 according to a protocol suitable for lower impedance tissue as explained below with reference to FIG. 6.

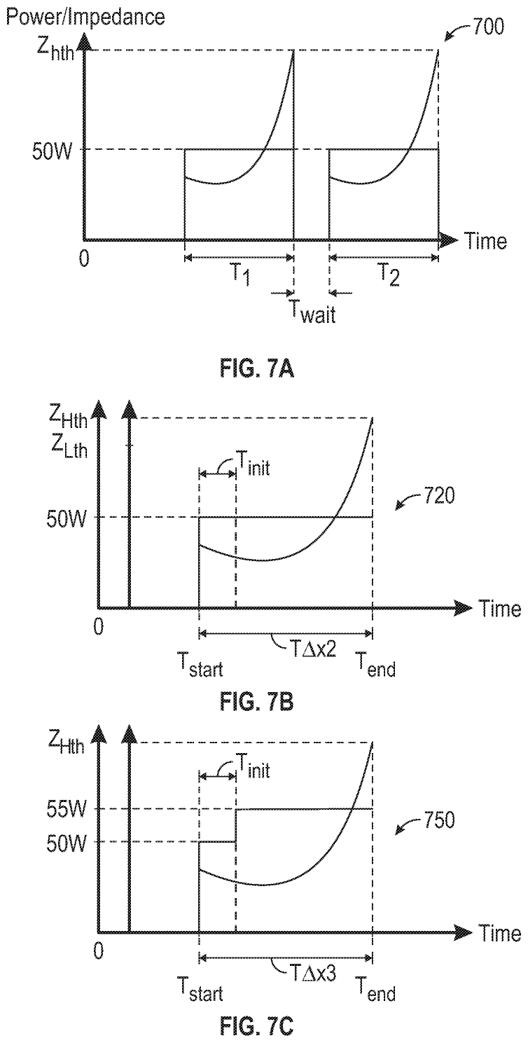

During operation 410, in response to decision operation 406 determining to use higher impedance tissue parameters during a latter portion of the tissue sealing process, the processor 122 executes instructions stored in memory 123 to cause the processor to provide control signals to the DC regulator 104 to cause the RF output stage 106 to imparts RF energy to tissue electrically coupled between the electrodes 108, 110 according to a protocol suitable for higher impedance tissue as explained below with reference to FIGS. 7A-7C.

FIG. 4B is a flowchart illustrating a method 450 of RF energy delivery during initial and latter portions of a biological tissue sealing process based upon phase angle between RF voltage and RF current during an initial portion of the sealing process. Operations 452, 458 and 460 of the method 450 of FIG. 4B correspond to operations 402. 408 and 410 of the method 400 of FIG. 4A In operation 454, the processor 122 executes instructions stored in memory 123 to cause the processor to determine phase angle between RF voltage and RF current delivered during the initial impedance measurement time window. Determining phase angle difference compensates for parasitic inductance and capacitance of the energy delivery network (cables and instrument), which can result in increased resolution to the measurement of change in impedance with respect to energy during the initial window portion in decision operation 456,

In decision operation 456, the processor 122 executes instructions stored in memory 123 to cause the processor to determine tissue type based upon measured average phase angle during the initial impedance measurement time window. An example method 450 determines tissue type according to rules set forth in the Table B.

TABLE B

| Average phase angle for initial | |
|---|---|
| impedance measurement time window | Parameters for latter portion of |
| of tissue sealing process | tissue sealing process |
| If avg phase angle in range <10 | Use low phase tissue parameters |
| If avg phase angle in range >10 | Use high phase tissue parameters |

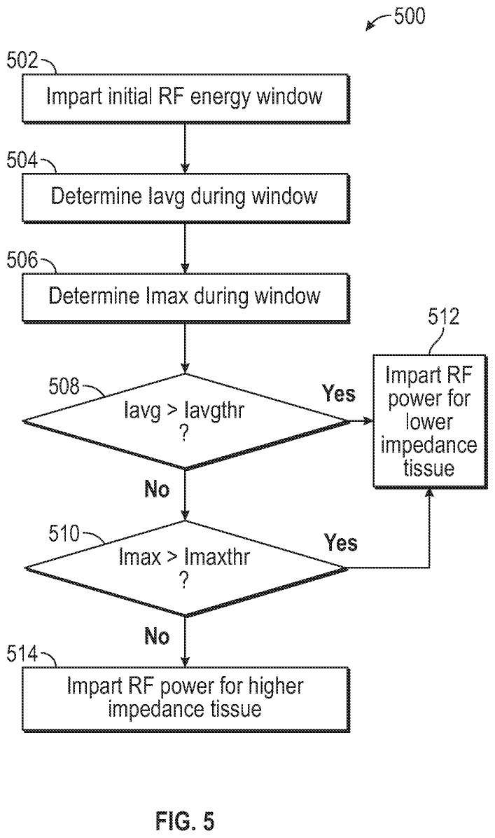

FIG. 5 is a flowchart illustrating operation of the electrosurgical generator system 100 in performing an example method 500 of controlling parameters for RF energy delivery during latter portion of a biological tissue sealing process based upon RF current flow through tissue during an initial portion of the sealing process. Operations in the method 500 may be performed using components of the electrosurgical generator system 100. As shown in FIG. 5, the method 500 includes operations 502-514.

In operation 502, the end effector 128 imparts RF energy to biological tissue during an initial current measurement time window, which has a duration in a range of 1 to 1000 ohms. The explanation of RF energy delivery is similar to that explained above for operation 402 of the method 400 of FIG. 4 and will not be repeated.

In operation 504, the processor 122 executes instructions stored in memory 123 to cause the processor to determine average current flow (I_{avg}) through the tissue during the initial current measurement time window. The processor 122 obtains RF current samples from the current sense transformer 116 and a second RMS converter 118, which monitor an RF output current between the first and second electrodes 108, 110 during the initial current measurement time window. Current flow through tissue is inversely proportional to impedance of the tissue. Thus, measurement of tissue current provides an alternative approach to determining tissue type based upon impedance. Moreover, average tissue current during the initial current measurement time window provides an indication of energy delivered to the tissue during that initial time window. Lower average current indicates lower energy delivery to the tissue during that initial time window, which indicates higher tissue impedance.

In operation 506, the processor 122 executes instructions stored in memory 123 to cause the processor to determine whether a maximum current (Iₘₐₓ) through the tissue during the initial current measurement time window indicates that the tissue is sealed during that initial window. The measurement of maximum current can avoid false positives that can result from reliance only upon average current measurement. For example, physical characteristics of a tissue sample captured between the jaws, such as dimensions, bulk and impedance may be such that sufficient energy is delivered to the sample during the initial current measurement time window to seal the sample. More particularly, physical characteristics of a tissue sample may be such that a sufficiently high current may flow through the tissue sample during the initial time window to deliver sufficient energy to seal the tissue during the initial time window. For example, a thinner tissue sample may seal during the initial time window. A lower impedance tissue sample that is relatively thin may seal during the initial time window. Once the tissue is sealed, impedance of the tissue decreases, resulting in decreased current flow, which may result in a lower average current during the initial time window. As explained herein, high impedance during initial time window generally indicates a need for delivery of added sealing energy delivery. A measure of maximum current during the initial window provides an indication of whether the tissue between the jaws is an exception to the rule, since high impedance is due to the tissue having been sealed already with no added sealing energy required.

In decision operation 508, the processor 122 executes instructions stored in memory 123 to cause the processor to determine tissue type based upon average current during the initial current measurement time window. In some examples of method 500, operation 508 determines whether average current meets a threshold. An example method 500 determines tissue type according to rules set forth in the Table C.

TABLE C

| I_{avg} for initial current measurement | Parameters for latter portion of |
|---|---|
| time window of tissue sealing process | tissue sealing process |
| If I_{avg} in a range >700 mA | Use lower impedance tissue |
| | parameters |
| IF I_{avg} in a range <700 mA | Use higher impedance tissue |
| | parameters (unless overruled by |
| | decision operation 510) |

It will be appreciated that decision operation 508 determines tissue type in terms of impedance based upon current flow characteristics of the tissue rather than precise identification of the biological constituents of the tissue. Different tissue samples may have different combinations of high and low impedance constituents. Rather than identify the specific tissue constituents, the operation 406 determines a measure of current flow indicative of impedance of a tissue portion captured between the jaws 204, 206.

In decision operation 510, the processor 122 executes instructions stored in memory 123 to cause the processor to determine whether tissue has been sealed during the initial time window based upon maximum current during the initial time window. In some examples of method 500, operation 510 determines whether maximum current meets a threshold. An example method 500 determines tissue type according to rules set forth in the Table D. It will be appreciated, of course, that the order in which decision operations 508, 510 is unimportant.

TABLE D

| Iₘₐₓ for initial current measurement | Parameters for latter portion of |
|---|---|
| time window of tissue sealing process | tissue sealing process |
| If Iₘₐₓ in a range >1000 mA | Use lower impedance tissue |
| | parameters |
| IF Iₘₐₓ in a range <1000 mA | Use higher impedance tissue |
| | parameters |

During operation 512, in response to decision operations 508 and 510 determining to use lower impedance tissue parameters during a latter portion of the tissue sealing process, the processor 122 executes instructions stored in memory 123 to cause the processor to provide control signals to the DC regulator 104 to cause the RF output stage 106 to imparts RF energy to tissue electrically coupled between the electrodes 108, 110 according to a protocol suitable for lower impedance tissue as explained below with reference to FIG. 6.

During operation 514, in response to decision operations 508 and 510 determining to use determining to use higher impedance tissue parameters during a latter portion of the tissue sealing process, the processor 122 executes instructions stored in memory 123 to cause the processor to provide control signals to the DC regulator 104 to cause the RF output stage 106 to imparts RF energy to tissue electrically coupled between the electrodes 108, 110 according to a protocol suitable for higher impedance tissue as explained below with reference to FIGS. 7A-7C.

FIGS. 6A-6B are illustrative example power delivery profiles 600, 650 representing alternative example power delivery profiles to control RF energy imparted through biological tissue in response to a determination during an initial window of a lower impedance tissue type. As used herein, the term ‘profile’ refers to RF energy signals delivered according to control algorithms stored as instructions in the memory 123 to configure the one or more processors 122 in controlling the sequence of operations (e.g., voltage and current pulses) to impart and halt energy to delivery tissue and to determine energy levels of the impulses. The timing diagrams of FIGS. 6A-6B represent alternative example implementations of operations 408, 458 and 512 of FIGS. 4A-4B and FIG. 5. The term, ‘lower impedance tissue type’ as used herein refers to impedance below 50 ohms or when impedance drops below 50 ohms during the initial time window.

Referring to FIG. 6A, the processor 122 causes the RF output stage 106 to generate an RF voltage sufficient to impart a substantially constant power, such as 50W for example, to the tissue during a fixed power delivery time interval T_{X1} that extends from Tₛₜₐᵣₜ to T_{end}. An example power delivery time T_{X1} falls within a time range 1 to 1000 ms. The processor 122 causes the RF output stage 106 to halt delivery of RF power to the tissue at the end of the sealing time interval T_{X1}.

Referring to FIG. 6B, the processor 122 causes the RF output stage 106 to generate an RF voltage sufficient to impart a substantially constant power, such as 50W for example, to the tissue during a power delivery time interval TΔ_{X1} that extends from Tₛₜₐᵣₜ to T_{end}. The processor 122 determines impedance characteristics of the tissue during an initial portion (Tᵢₙᵢₜ) of the power delivery time interval TΔ_{X1} as explained above with reference to one of the methods 400, 450, 500 described above with reference to FIGS. 4A-4B and FIG. 5. An example power delivery time Tᵢₙᵢₜ falls within a time range 1 to 1000 ms. During a latter portion of the power delivery time interval TΔ_{X1} that extends from the end of the initial time interval Tᵢₙᵢₜ to the end time T_{end}, the processor 122 monitors impedance of the tissue based upon voltage and current information provided by the RMS voltage converter 114 and the RMS current converter 118. The processor 122 causes the RF output stage 106 to halt delivery of RF power in response to the tissue impedance reaching a predetermined impedance threshold Z_{Hth} for a lower impedance tissue. In an example it will be appreciated that duration of the power delivery time TΔ_{X1} varies depending upon how much time is required for tissue impedance to reach Z_{Hth}.

FIGS. 7A-7C arc illustrative alternative example timing diagrams representing alternative example power delivery profiles 700, 720, 750 to control RF energy imparted through biological tissue in response to determination during an initial window of a higher impedance tissue type. The timing diagrams 600 of FIGS. 7A-7C represent alternative example implementations of the operations 410, 460 and 514 of FIGS. 4A-4B and FIG. 5. During each of the power delivery profiles of FIGS. 7A-7C, the processor 122 determines impedance characteristics of the tissue during an initial time window (Tᵢₙᵢₜ) as explained above. The term ‘higher impedance tissue type’ as used herein refers to impedance starting at higher than 200 ohms or not falling below 100 ohms within Tᵢₙᵢₜ window.

Referring to FIG. 7A, during the initial time window, T₁, the processor 122 executes instructions stored in memory 123 to cause the processor to cause the RF output stage 106 to generate a first voltage signal sufficient to deliver a first power level, such as 50W for example, to the tissue. In response to determination during the initial time window T₁ that the tissue has a higher impedance type, the processor 122 monitors impedance of the tissue based upon voltage and current information provided by the RMS voltage converter 114 and the RMS current converter 118. The processor 122 executes instructions stored in memory 123 to cause the processor to cause the RF output stage 106 to halt delivery of RF power in response to the tissue impedance reaching a predetermined impedance threshold Z_{Hth} for a higher impedance tissue. The processor 122 then waits for a T_{wait} time then executes instructions stored in memory 123 to cause the processor to cause the RF output stage 106 to generate a second voltage signal sufficient during a second time window T₂, to deliver a power level, such as 50W for example, to the tissue. The processor 122 monitors impedance of the tissue based upon voltage and current information provided by the RMS voltage converter 114 and the RMS current converter 118. The processor 122 executes instructions stored in memory 123 to cause the processor to cause the RF output stage 106 to halt delivery of RF power in response to the tissue impedance reaching a predetermined impedance threshold Z_{Hth} for a higher impedance tissue. The delivery of two consecutive RF power leads to a better seal on higher impedance tissue. The time delay T_{wait} rests the tissue between voltage signals to avoid unwanted tissue damage.

Referring to FIG. 79, during a latter portion of a power delivery time interval TΔ_{X2} that extends from the end of the initial time interval Tᵢₙᵢₜ to the end time T_{end}, the processor 122 monitors impedance of the tissue based upon voltage and current information provided by the RMS voltage converter 114 and the RMS current converter 118. The processor 122 executes instructions stored in memory 123 to cause the processor to cause the RF output stage 106 to halt delivery of RF power in response to the tissue impedance reaching a predetermined impedance threshold Z_{Hth} for a higher impedance tissue.

Referring to FIG. 7C, during the initial time window, Tᵢₙᵢₜ, the processor 122 executes instructions stored in memory 123 to cause the processor to cause the RF output stage 106 to generate a voltage sufficient to deliver a first lower power level, such as 50W for example, to the tissue. In response to determination during the initial time window that the tissue has a higher impedance type, the processor 122, during a latter portion of a power delivery time interval TΔ_{X3}, causes the RF output stage 106 to generate a voltage sufficient to deliver a second higher power level, such as 55W for example, to the tissue. During the latter part of the power delivery time interval TΔ_{X3}, the processor 122 monitors impedance of the tissue based upon voltage and current information provided by the RMS voltage converter 114 and the RMS current converter 118. The processor 122 causes the RF output stage 106 to halt delivery of RF power in response to the tissue impedance reaching a predetermined impedance threshold Z_{Hth} for a higher impedance tissue. The delivery of a higher is RF power level during the latter part of the tissue scaling stage reduces the overall time required to seal a higher impedance tissue.

### EXAMPLE 1

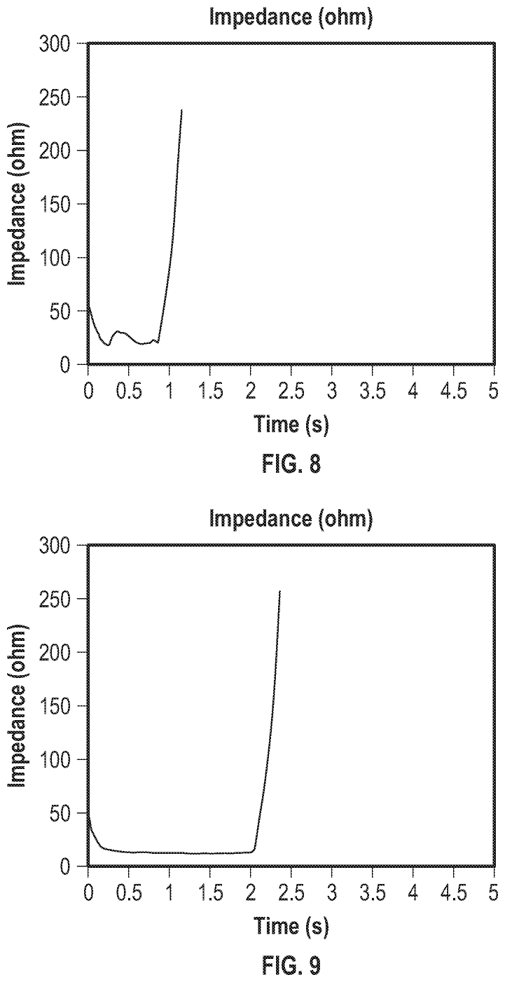

FIG. 8 is an illustrative example plot showing tissue impedance versus time during RF power delivery to a lower impedance artery tissue. Assume that the initial time window (Tᵢₙᵢₜ) is one second in duration and extends from 0 seconds to 1 second. Impedance begins a steep rise during the initial time interval. The processor 122 monitors impedance versus energy delivery during the initial time window to determine that the tissue is lower impedance type as explained above.

### EXAMPLE 2

FIG. 9 is an illustrative example plot showing tissue impedance versus time during RF power delivery to a higher impedance thin mesentery tissue. Assume that the initial time window (Tᵢₙᵢₜ) is one second in duration and extends from 0 seconds to 1 second. Impedance remains substantially flat and does not begin a steep rise during the initial time interval. The processor 122 monitors impedance versus energy delivery during the initial time window to determine that the tissue is higher impedance type as explained above.

### EXAMPLE 3

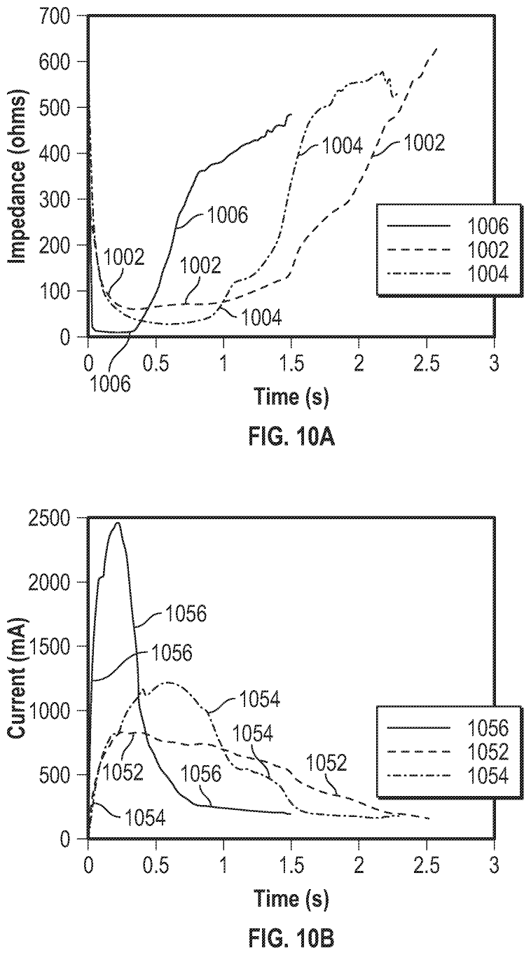

FIGS. 10A-10B are illustrative plots showing impedance versus time (FIG. 10A) and current versus time (FIG. 10B) during RF power delivery to a three different impedance type tissues. Assume that the initial time window (Tᵢₙᵢₜ) is one second in duration and extends from 0 to 1 second.

Referring to FIGS. 110A-1B, a first impedance plot 1002 of FIG. 10A and a first current plot 1052 of FIG. 10B correspond to a first high impedance tissue, such as tissue 300 of FIG. 3A. Referring to FIG. 10A, the processor 122 is configured in one example, to determine that the first tissue, which does not have a significant impedance rise during the initial time window, is a high impedance type based upon one of the example methods of FIGS. 4A-4B. Referring to FIG. 10B, the processor 122 is configured in another example, to determine that the first tissue, which has a low maximum current (˜800 mA) and low average current (˜600 mA) during the initial time window, is a high impedance type based upon the example method 500 of FIG. 5.

Referring to FIGS. 10A-10B, a second impedance plot 1004 of FIG. 10A and a second current plot 1054 of FIG. 10B correspond to a second high impedance tissue, such as tissue 310 of FIG. 3B. Referring to FIG. 10A, the processor 122 is configured in one example, to determine that the second tissue, which has a moderate impedance rise during the initial time window, is a moderate impedance type based upon one of the example methods of FIGS. 4A-4B. Referring to FIG. 10B, the processor 122 is configured in another example, to determine that the second tissue, which has a normal peak current (>1000 mA) and normal average current (˜900 mA) during the initial time window, is a moderate impedance type based upon the example method 500 of FIG. 5.

Referring to FIGS. 10A-10B, a third impedance plot 1006 of FIG. 10A and a third current plot 1056 of FIG. 10B correspond to a third high impedance tissue, such as tissue 320 of FIG. 3C. Referring to FIG. 10A, the processor 122 is configured in one example, to determine that the third tissue, which has a steep impedance rise during the initial time window, is a lower impedance type based upon one of the example methods of FIGS. 4A-4B. Referring to FIG. 10B, the processor 122 is configured in another example, to determine that the third tissue, which has a high peak current (>2000 mA), but low average current (˜500 mA) during the initial time window, is a lower impedance type based upon the example method 500 of FIG. 5.

### Various Examples

Example 1 includes an electrosurgical method to seal biological tissue comprising: imparting radio frequency (RF) power to biological tissue during an RF power delivery time interval; measuring a change in impedance of the tissue with respect to RF energy delivered to the tissue during an initial portion of the RF power delivery time interval; selecting an RF power delivery profile to impart RF power to the tissue during a latter portion of the RF power delivery time interval following the initial portion, based at least in part upon the measured change in impedance of the tissue with respect to RF energy delivered to the tissue during the initial portion; and imparting RF power to the tissue according to the selected RF power delivery profile during the latter portion of the RF power delivery time interval.

Example 2 can include the subject matter of Example 1 wherein selecting the RF power delivery profile includes, selecting an RF power delivery profile to deliver a first amount of energy in the latter portion of the RF power delivery time interval in response to a lower measured change in impedance with respect to RF energy delivered during the initial portion; and selecting an RF power delivery profile to deliver a second amount of energy in the latter part of the RF power delivery time interval in response to higher measured change in impedance with respect to RF energy delivered during the initial portion; wherein the first amount of energy is greater than the second amount of energy.

Example 3 can include the subject matter of Example 1 wherein selecting the RF power delivery profile includes, selecting a higher RF power during the latter portion of the RF power delivery time interval in response to a lower measured change in impedance with respect to RF energy delivered; and selecting a lower RF power during the latter portion of the RF power delivery time interval in response to a higher measured change in impedance with respect to RF energy delivered.

Example 4 can include the subject matter of Example 1 wherein selecting the RF power delivery profile includes, selecting a longer latter portion of the RF power delivery time interval in response to a lower measured change in impedance with respect to RF energy delivered; and selecting a shorter latter portion of the RF power delivery time interval in response to a higher measured change in impedance with respect to RF energy delivered.

Example 5 can include the subject matter of Example 1 further including: monitoring impedance of the tissue during the latter portion of the RF power delivery time interval; and halting delivery of RF power in response to impedance of the tissue reaching an impedance threshold value during the latter portion of the RF power delivery time interval.

Example 6 can include the subject matter of Example 1 wherein selecting the RF power delivery profile includes, selecting a higher impedance threshold value in response to a lower measured change in impedance with respect to RF energy delivered; and selecting a lower impedance threshold value in response to a higher measured change in impedance with respect to RF energy delivered.

Example 7 can include the subject matter of Example 1 wherein selecting the RF power delivery profile includes, selecting a higher impedance threshold value and a higher RF power during the latter portion of the RF power delivery time interval in response to a lower measured change in impedance with respect to RF energy delivered; and selecting a lower impedance threshold value and a lower RF power during the latter portion of the RF power delivery time interval in response to a higher measured change in impedance with respect to RF energy delivered.

Example 8 can include the subject matter of Example 1 wherein measuring a change in impedance of the tissue with respect to RF energy delivered to the tissue during an initial portion of the RF power delivery time interval includes totalizing power during the initial portion.

Example 9 can include the subject matter of Example 1 wherein imparting RF power to biological tissue during the RF power delivery time interval include delivering a predetermined RF power during the initial portion.

Example 10 can include the subject matter of Example 1 wherein measuring a change in impedance of the tissue with respect to RF energy delivered to the tissue during an initial portion of the RF power delivery time interval includes measuring RF voltage across the tissue and measuring RF current through the tissue.

Example 11 can include the subject matter of Example 1 wherein measuring change in impedance of the tissue includes measuring phase angle between RF voltage across the tissue to RF current through the tissue.

Example 12 includes an electrosurgical system to seal biological tissue comprising: imparting radio frequency (RF) power to biological tissue during a first RF power delivery time interval, measuring a change in impedance of the tissue with respect to RF energy delivered to the tissue during an initial portion of the first RF power delivery time interval; in response to measured tissue impedance reaching the predetermined impedance threshold for a high impedance tissue during the initial portion, halting delivery of RF power during the initial time interval of the first RF power delivery time interval in response to measured tissue impedance reaching a predetermined impedance threshold for a high impedance tissue during the initial portion; and imparting RF power to the tissue during a second RF power delivery time interval, after a predetermined delay following the halting; in response to measured tissue impedance not reaching the predetermined impedance threshold for a high impedance tissue during the initial portion, continuing to impart RF power during a latter part of the first RF power delivery window.

Example 13 can include the subject matter of Example 12 comprising: an RF output stage configured to impart an RF power to the tissue; current measurement circuitry configured to measure RF current within the tissue during the imparting of the RF power to the tissue; voltage measurement circuitry configured to measure of RF voltage across the tissue during the imparting of the RF power to the tissue; a processor circuit; a memory storing instructions that, when executed by the processor, cause the processor to perform operations including: causing the RF output stage to impart RF power to the tissue during an RF power delivery time interval; determining a change in impedance of the tissue based upon current measured by the current measurement circuit and voltage measured by the voltage measurement circuit during an initial portion of the RF power delivery time interval; determining an indication of RF energy delivered to the tissue during the initial portion based upon at least one of totalizing RF power delivered to the tissue during the initial portion, and a duration of the initial portion; selecting an RF power delivery profile to impart RF power to the tissue during; a latter portion of the RF power delivery time interval following the initial portion, based at least in part upon the measured change in impedance of the tissue with respect to the indicated RF energy delivered to the tissue during the initial portion and causing the RF output stage to impart RF power to the tissue according to the selected RF power delivery profile during the latter portion of the RF power delivery time interval.

Example 14 can include the subject matter of Example 13 wherein selecting the RF power delivery profile includes, selecting an RF power delivery profile to deliver a first amount of energy in the latter portion of the RF power delivery time interval in response to a lower measured change in impedance with respect to the indicated RF energy delivered during the initial portion; and selecting an RF power delivery profile to deliver a second amount of energy in the latter part of the RF power delivery time interval in response to higher measured change in impedance with respect to the indicated RF energy delivered during the initial portion; wherein the first amount of energy is greater than the second amount of energy.

Example 15 can include the subject matter of Example 13 wherein selecting the RF power delivery profile includes, selecting a higher RF power during the latter portion of the RF power delivery time interval in response to a lower measured change in impedance with respect to the indicated RF energy delivered; and selecting a lower RF power during the latter portion of the RF power delivery time interval in response to a higher measured change in impedance with respect to the indicated RF energy delivered.

Example 16 can include the subject matter of Example 13 wherein selecting the RF power delivery profile includes, selecting a longer latter portion of the RF power delivery time interval in response to a lower measured change in impedance with respect to the indicated RF energy delivered; and selecting a shorter latter portion of the RF power delivery time interval in response to a higher measured change in impedance with respect to the indicated RF energy delivered.

Example 17 can include the subject matter of Example 13 wherein the operations further including: monitoring impedance of the tissue during the latter portion of the RF power delivery time interval; and halting delivery of RF power in response to impedance of the tissue reaching an impedance threshold value during the latter portion of the RF power delivery time interval.

Example 18 can include the subject matter of Example 1 wherein selecting the RF power delivery profile includes, selecting a higher impedance threshold value in response to a lower measured change in impedance with respect to the indicated RF energy delivered; and selecting a lower impedance threshold value in response to a higher measured change in impedance with respect to the indicated RF energy delivered.

Example 19 can include the subject matter of Example 17 wherein selecting the RF power delivery profile includes, selecting a higher impedance threshold value and a higher RF power during the latter portion of the RF power delivery time interval in response to a lower measured change in impedance with respect to RF energy delivered; and selecting a lower impedance threshold value and a lower RF power during the latter portion of the RF power delivery time interval in response to a higher measured change in impedance with respect to the indicated RF energy delivered.

Example 20 includes an electrosurgical method to seal biological tissue comprising: imparting RF power to biological tissue during an RF power delivery time interval; measuring RF current within the tissue during an initial portion of the RF power delivery time interval; selecting an RF power delivery profile to impart RF power to the tissue during a latter portion of the RF power delivery time interval following the initial portion, based at least in part upon the measured RF current within the tissue during the initial portion; and imparting RF power to the tissue according to the selected RF power delivery profile during the latter portion of the RF power delivery time interval.

Example 21 can include the subject matter of Example 20 wherein selecting the RF power delivery profile includes, selecting an RF power delivery profile to deliver a first amount of energy in the latter portion of the RF power delivery time interval in response to a measured RF current within the tissue indicative of a lower impedance of the tissue; and selecting art RF power delivery profile to deliver a second amount of energy in the latter part of the RF power delivery time interval in response to a measured RF current within the tissue indicative of a higher impedance of the tissue; wherein the first amount of energy is greater than the second amount of energy.

Example 22 can include the subject matter of Example 20 wherein measuring RF current within the tissue during an initial portion of the RF power delivery time interval includes, measuring at least one of average RF current within the tissue and integral of RF current within the tissue; and measuring maximum RF current within the tissue.

Example 23 can include the subject matter of Example 20 wherein measuring RF current within the tissue during an initial portion of the RF power delivery time interval includes, measuring at least one of average RF current within the tissue and integral of RF current within the tissue, and measuring maximum RF current within the tissue; and wherein selecting the RF power delivery profile includes, selecting an RF power delivery profile to deliver a first amount of energy in the latter portion of the RF power delivery time interval in response to an occurrence of at least one of, measured maximum RF current below a maximum current threshold and at least one of measured average current meeting an average current threshold, and at least one of, measured average current meeting an average current threshold and measured integral of current meeting an integral of current threshold, and selecting an RF power delivery profile to deliver a second amount energy in the latter portion of the RF power delivery time interval in response to an occurrence of, at least one of, measured average current meeting an average current threshold and measured integral of current meeting an integral of current threshold; wherein the first amount of energy is greater than the second amount of energy.

Example 24 includes an electrosurgical method to seal biological tissue comprising: an RF output stage configured to impart an RF power to the tissue; current measurement circuitry configured to measure RF current within the tissue during the imparting of the RF power to the tissue; a processor circuit; a memory storing instructions that, when executed by the processor, cause the processor to perform operations including: causing the RF output stage to impart RF power to the tissue during an RF power delivery time interval; selecting an RF power delivery profile to impart RF power to the tissue during a latter portion of the RF power delivery time interval following the initial portion, based at least in part upon the RF current measured by the current measurement circuitry during an initial portion RF power delivery time interval; and causing the RF output stage to impart RF power to the tissue according to the selected RF power delivery profile during the latter portion of the RF power delivery time interval.

Example 25 can include the subject matter of Example 24 wherein selecting an RF power delivery profile includes, selecting an RF power delivery profile to deliver a first amount of energy in the latter portion of the RF power delivery time interval in response to a measured RF current within the tissue indicative of a lower impedance of the tissue; and selecting an RF power delivery profile to deliver a second amount of energy in the latter part of the RF power delivery time interval in response to a measured RF current within the tissue indicative of a higher impedance of the tissue; wherein the first amount of energy is greater than the second amount of energy.

Example 26 can include the subject matter of Example 24 wherein selecting an RF power delivery profile includes, selecting an RF power delivery profile to deliver a first amount of energy in the latter portion of the RF power delivery time interval in response to an occurrence of at least one of, measured maximum RF current below a maximum current threshold and at least one of measured average current meeting an average current threshold, and at least one of, measured average current meeting an average current threshold and measured integral of current meeting an integral of current threshold, and selecting an RF power delivery profile to deliver a second amount energy in the latter portion of the RF power delivery time interval in response to an occurrence of, at least one of, measured average current meeting an average current threshold and measured integral of current meeting an integral of current threshold; wherein the first amount of energy is greater than the second amount of energy.

The above description is presented to enable any person skilled in the art to create and use a system and method to control RF energy delivery to seal biological tissue based upon at least one of impedance or current during an initial time window. Various modifications to the examples will be clear to those skilled in the art, and the generic principles defined herein may be applied to other examples and applications without departing from the scope of the invention. In the preceding description, numerous details are set forth for explanation. However, one of ordinary skill in the art will realize that the circuitry might be practiced without the use of these specific details. In other instances, well-known circuits and processes are shown in block diagram form in order not to obscure the description of the invention with unnecessary detail. Identical reference numerals may be used to represent different views of the same or similar item in different drawings and in the specification. Thus, the foregoing description and drawings of examples in accordance with the present invention are merely illustrative of the principles of the invention. Therefore, it will be understood that various modifications can be made to the examples by those skilled in the art without departing from the scope of the invention, which is defined in the appended claims.

1. An electrosurgical method to seal biological tissue comprising:
imparting radio frequency (RF) power to biological tissue during an RF power delivery time interval;
measuring a change in impedance of the tissue with respect to RF energy delivered to the tissue during an initial portion of the RF power delivery time interval;
selecting an RF power delivery profile to impart RF power to the tissue during a latter portion of the RF power delivery time interval following the initial portion, based at least in part upon the measured change in impedance of the tissue with respect to RF energy delivered to the tissue during the initial portion; and
imparting RF power to the tissue according to the selected RF power delivery profile during the latter portion of the RF power delivery time interval.

2. The method of claim 1,
wherein selecting the RF power delivery profile includes,
selecting an RF power delivery profile to deliver a first amount of energy in the latter portion of the RF power delivery time interval in response to a lower measured change in impedance with respect to RF energy delivered during the initial portion; and
selecting an RF power delivery profile to deliver a second amount of energy in the latter part of the RF power delivery time interval in response to higher measured change in impedance with respect to RF energy delivered during the initial portion;
wherein the first amount of energy is greater than the second amount of energy.

3. The method of claim 1,
wherein selecting the RF power delivery profile includes,
selecting a higher RF power during the latter portion of the RF power delivery time interval in response to a lower measured change in impedance with respect to RF energy delivered; and
selecting a lower RF power during the latter portion of the RF power delivery time interval in response to a higher measured change in impedance with respect to RF energy delivered. cm 4. The method of claim 1,
wherein selecting the RF power delivery profile includes,
selecting a longer latter portion of the RF power delivery time interval in response to a lower measured change in impedance with respect to RF energy delivered; and.
selecting a shorter latter portion of the RF power delivery time interval in response to a higher measured change in impedance with respect to RF energy delivered.

5. The method of claim 1 further including:
monitoring impedance of the tissue during the latter portion of the RF power delivery time interval; and
halting delivery of RF power in response to impedance of the tissue reaching an impedance threshold value during the latter portion of the RF power delivery time interval.

6. The method of claim 5,
wherein selecting the RF power delivery profile includes,
selecting a higher impedance threshold value in response to a lower measured change in impedance with respect to RF energy delivered; and
selecting a lower impedance threshold value in response to a higher measured change in impedance with respect to RF energy delivered.

7. The method of claim 5,
wherein selecting the RF power delivery profile includes,
selecting a higher impedance threshold value and a higher RF power during the latter portion of the RF power delivery time interval in response to a lower measured change in impedance with respect to RF energy delivered; and
selecting a lower impedance threshold value and a lower RF power during the latter portion of the RF power delivery time interval in response to a higher measured change in impedance with respect to RF energy delivered.

8. The method of claim 1,
wherein measuring a change in impedance of the tissue with respect to RF energy delivered to the tissue during an initial portion of the RF power delivery time interval includes totalizing power during the initial portion.

9. The method of claim 1,
wherein imparting RF power to biological tissue during the RF power delivery time interval include delivering a predetermined RF power during the initial portion.

10. The method of claim 1,
wherein measuring a change in impedance of the tissue with respect to RF energy delivered to the tissue during an initial portion of the RF power delivery time interval includes measuring RF voltage across the tissue and measuring RF current through the tissue.

11. The method of claim 1,
wherein measuring change in impedance of the tissue includes measuring phase angle between RF voltage across the tissue to RF current through the tissue.

12. An electrosurgical method to seal biological tissue comprising:
imparting radio frequency (RF) power to biological tissue during a first RF power delivery time interval;
measuring a change in impedance of the tissue with respect to RF energy delivered to the tissue during an initial portion of the first RF power delivery time interval;
in response to measured tissue impedance reaching the predetermined impedance threshold for a high impedance tissue during the initial portion,
halting delivery of RF power during the initial time interval of the first RF power delivery time interval in response to measured tissue impedance reaching a predetermined impedance threshold for a high impedance tissue during the initial portion; and
imparting RF power to the tissue during a second RF power delivery time interval, after a predetermined delay following the halting;
in response to measured tissue impedance not reaching the predetermined impedance threshold for a high impedance tissue during the initial portion,
continuing to impart RF power during a latter part of the first RF power delivery window.

13. An electrosurgical system for sealing biological tissue, comprising:
an RF output stage configured to impart an RF power to the tissue;
current measurement circuitry configured to measure RF current within the tissue during the imparting of the RF power to the tissue;
voltage measurement circuitry configured to measure of RF voltage across the tissue during the imparting of the RF power to the tissue;
a processor circuit;
a memory storing instructions that, when executed by the processor, cause the processor to perform operations including:
causing the RF output stage to impart RF power to the tissue during an RF power delivery time interval;
determining a change in impedance of the tissue based upon current measured by the current measurement circuit and voltage measured by the voltage measurement circuit during an initial portion of the RF power delivery time interval;
determining an indication of RF energy delivered to the tissue during the initial portion based upon at least one of totalizing RF power delivered to the tissue during the initial portion, and a duration of the initial portion;
selecting an RF power delivery profile to impart RF power to the tissue during a latter portion of the RF power delivery time interval following the initial portion, based at least in part upon the measured change in impedance of the tissue with respect to the indicated RF energy delivered to the tissue during the initial portion; and
causing the RF output stage to impart RF power to the tissue according to the selected RF power delivery profile during the latter portion of the RF power delivery time interval.

14. The system of claim 13,
wherein selecting the RF power delivery profile includes,
selecting an RF power delivery profile to deliver a first amount of energy in the latter portion of the RF power delivery time interval in response to a lower measured change in impedance with respect to the indicated RF energy delivered during the initial portion; and
selecting an RF power delivery profile to deliver a second amount of energy in the latter part of the RF power delivery time interval in response to higher measured change in impedance with respect to the indicated RF energy delivered during the initial portion;
wherein the first amount of energy is greater than the second amount of energy.

15. The system of claim 13,
wherein selecting the RF power delivery profile includes,
selecting a higher RF power during the latter portion of the RF power delivery time interval in response to a lower measured change in impedance with respect to the indicated RF energy delivered; and
selecting a lower RF power during the latter portion of the RF power delivery time interval in response to a higher measured change in impedance with respect to the indicated RF energy delivered.

16. The system of claim 13,
wherein selecting the RF power delivery profile includes,
selecting a longer latter portion of the RF power delivery time interval in response to a lower measured change in impedance with respect to the indicated RF energy delivered; and
selecting a shorter latter portion of the RF power delivery time interval in response to a higher measured change in impedance with respect to the indicated RF energy delivered.

17. The system of claim 13,
wherein the operations further including:
monitoring impedance of the tissue during the latter portion of the RF power delivery time interval; and
halting delivery of RF power in response to impedance of the tissue reaching an impedance threshold value during the latter portion of the RF power delivery time interval.

18. The system of claim 17,
wherein selecting the RF power delivery profile includes,
selecting a higher impedance threshold value in response to a lower measured change in impedance with respect to the indicated RF energy delivered; and
selecting a lower impedance threshold value in response to a higher measured change in impedance with respect to the indicated RF energy delivered.

19. The system of claim 17,
wherein selecting the RF power delivery profile includes,
selecting a higher impedance threshold value and a higher FF power during the latter portion of the RF power delivery time interval in response to a lower measured change in impedance with respect to RF energy delivered; and
selecting a lower impedance threshold value and a lower RF power during the latter portion of the RF power delivery time interval in response to a higher measured change in impedance with respect to the indicated RF energy delivered.

20-26. (canceled)
